(19) United States
(12) Patent Application Publication
(10) Pub. No.: US 2011/0288500 A1
(43) Pub. Date: Nov. 24, 2011

(54) DRUG-DELIVERY ELEMENT FOR AN ELONGATE IMPLANTABLE MEDICAL DEVICE COMPONENT

(76) Inventors: Fysh Dadd, Lane Cove (AU); Jane L. Rapsey, Berowra (AU); Peter Gibson, South Coogee (AU); Peter Schuller, Turramurra (AU); Carrie Newbold, Richmond (AU)

(21) Appl. No.: 12/786,320

(22) Filed: May 24, 2010

(51) Int. Cl. A61L 31/16; B32B 37/12

(52) U.S. Cl. 604/275; 156/294

(57) ABSTRACT

An implantable medical device including an implantable elongate component and at least one elongate drug-delivery element. The elongate drug-delivery element releasably carries at least one drug and is configured to be mounted to the elongate component such that the longitudinal axis of the drug-delivery element is substantially parallel with a longitudinal axis of the elongate component.

### BACKGROUND

1. Field of the Invention

The present invention relates generally to an implantable medical device, and more particularly, to a drug-delivery element for an elongate implantable medical device component.

2. Related Art

Medical devices having one or more implantable components, generally referred to herein as implantable medical devices, have provided a wide range of therapeutic benefits to patients (sometimes referred to herein as recipients) over recent decades. Some implantable medical devices include an implantable elongate component that itself performs a therapeutic function, or serves as a carrier for devices that perform such function. Such devices include, for example, devices that perform imaging, detection of physiological conditions, delivery of drugs, application of electrical stimulation, and so on. For example, cochlear implants include an elongate component configured to apply stimulation to a recipient and/or receive signals from a recipient's tissue.

Traditionally, there has been interest in delivering bioactive substances or chemicals (generally and collectively referred to herein as “drugs”) in conjunction with a cochlear implant and other implantable medical device. In one conventional drug delivery approach, the implantable medical device is coated with a bioactive substance. In another conventional approach, a bioactive substance is integrated into the polymeric coating of the implantable medical device. These and other conventional approaches typically require the incorporation of the drug into the implantable medical device during the manufacturing process of the device. This introduces a number of difficult problems and challenges for the manufacturing and sterilization processes, particularly for complex implantable medical devices. In other conventional drug delivery approaches, liquid drugs are contained in an external or implanted reservoir and are transferred to a target location in a patient.

### SUMMARY

In one aspect of the present invention, an implantable medical device is provided. The device comprises an implantable elongate component, and at least one elongate drug-delivery element releasably carrying at least one drug and configured to be mounted to the elongate component such that the longitudinal axis of the drug-delivery element is substantially parallel with a longitudinal axis of the elongate component.

In another aspect of the present invention, a kit for an implantable drug-delivery system is provided. The kit comprises an implantable elongate component, at least one first drug-delivery element, and at least one second drug-delivery element. Each of the first and second drug-delivery members releasably carries at least one drug and is configured to be mounted along a length of the elongate component such that the longitudinal axis of the drug-delivery element is substantially parallel with a longitudinal axis of the elongate component.

In another aspect of the present invention, an implantable elongate drug-delivery element for a distally extending implantable elongate component of an implantable medical device is provided. The elongate component comprises an elongate recess. The drug-delivery element comprises an elongate substrate configured to be releasably mounted in the elongate recess such that the longitudinal axis of the drug-delivery element is substantially parallel with the longitudinal axis of the elongate component, and a drug releasably carried by the substrate.

In another aspect of the present invention, a method of using an implantable medical device comprising a distally extending implantable elongate component and at least one drug-delivery element releasably carrying at least one drug is provided. The method comprises mounting the drug-delivery element along a length of the elongate component such that the longitudinal axis of the drug-delivery element is substantially parallel with a longitudinal axis of the elongate component, and implanting the elongate component in a recipient subsequent to mounting the drug-delivery member.

### BRIEF DESCRIPTION OF THE DRAWINGS

Illustrative embodiments of the present invention are described herein with reference to the accompanying drawings, in which:

FIG. 1A is a perspective view of an exemplary cochlear implant with which a drug-delivery element in accordance with embodiments of the present invention may be implemented;

FIG. 1B is a side view of a portion of an exemplary drug-delivery accessory, in accordance with embodiments of the present invention;

FIG. 2A is a side view of a portion of an implantable medical device having an elongate drug-delivery element mounted on an implantable elongate component of the device, in accordance with embodiments of the present invention

FIG. 2B is a cross-sectional view of the stimulating assembly of FIG. 2A taken along section line 2B-2B of FIG. 2A in a proximal region, in accordance with embodiments of the present invention;

FIG. 2C is a cross-sectional view of the stimulating assembly of FIG. 2A and elongate drug-delivery spine taken along section line 2C-2C of FIG. 2A in a drug-delivery region, in accordance with embodiments of the present invention;

FIG. 3A is a top view of a portion of an implantable medical device having an elongate drug-delivery element mounted on an implantable elongate component of the device, in accordance with embodiments of the present invention;

FIG. 3B is a cross-sectional view of the stimulating assembly of FIG. 3A and the elongate drug delivery spine of FIG. 3A taken along section line 3B-3B of FIG. 3A, in accordance with embodiments of the present invention;

FIGS. 4A-4C are cross-sectional views of alternate embodiments of the stimulating assembly and elongate drug-delivery spine of FIGS. 3A and 3B, in accordance with embodiments of the present invention;

FIG. 5A is a side perspective view of a portion of an implantable medical device having an elongate drug-delivery element that mechanically locks into a recess in an implantable elongate component of the device, in accordance with embodiments of the present invention;

FIG. 5B is a cross-sectional view of the stimulating assembly of FIG. 5A when the spine of FIG. 5A is mounted on the stimulating assembly, in accordance with embodiments of the present invention;

FIG. 6A is a side perspective view of a portion of an implantable medical device having an implantable elongate component and an elongate drug-delivery element that may be mounted on an implantable elongate component of the device, in accordance with embodiments of the present invention;

FIG. 6B is a cross-sectional view of a stimulating assembly illustrated in FIG. 6A along the plane illustrated in FIG. 6A when the drug-delivery element of FIG. 6A is mounted to the elongate component;

FIG. 7A is a top view of a portion of an implantable medical device having a plurality of elongate drug-delivery elements mounted on an implantable elongate component of the device, in accordance with embodiments of the present invention; and

FIG. 7B is a top view of a portion of an implantable medical device having an elongate drug-delivery element mounted on an implantable elongate component of the device, in accordance with embodiments of the present invention.

### DETAILED DESCRIPTION

The present invention is directed to an elongate drug-delivery element for an implantable medical device having one or more components. Embodiments of the present invention include an elongate drug-delivery element releasably carrying (e.g., covered with, impregnated with, etc.) one or more drugs. Aspects of the present invention are directed to an elongate drug-delivery element for an implantable elongate carrier member, lead, catheter or the like (collectively and generally referred to as an “elongate component” or “carrier member”) of an implantable medical device. As used herein, an “elongate” element or component is an element or component having a length that is substantially greater than its width and its height.

Embodiments of the elongate drug-delivery element are physically separate from the implantable medical device having one or more implantable components. As such, the drug-delivery element may be manufactured separately from the device components, which is referred to herein as being “separate” from the medical device. The drug-delivery element is operationally combined with a component of the implantable medical device subsequent to the device's manufacture and/or sterilization. Embodiments of the drug-delivery element may be configured, for example, to attain an implanted position adjacent to one or more surfaces of an implantable medical device component. In certain embodiments, the element is configured in the form of a spine, rod, layer, or the like (collectively and generally referred to as a “spine” herein), to be mounted along a length of the elongate component such that the longitudinal axis of the drug-delivery element is substantially parallel with a longitudinal axis of the elongate component. Embodiments of the drug delivery accessory may be implanted into the recipient concurrently with the implantation of the implantable medical device. As used herein, the term “implantable medical device” encompasses both partially implantable and fully implantable medical devices.

Providing an independently-manufactured and physically distinct elongate drug-delivery element to an implantable medical device increases flexibility in an applied therapy while reducing the undesirable aspects associated with manufacturing an implantable medical device with a drug integrated therein. At least some conventional medical device manufacturing processes include applying a drug to a component of the medical device. In one example, a drug is applied to a cochlear implant during the manufacturing process for the cochlear implant. In some conventional applications, a drug carried by the cochlear implant may be released via elution or through resorption of a feature carrying the drug. In these applications, the elution and the resorption are each triggered by interaction with moisture. In an intra-cochlear environment, the triggering moisture is perilymph fluid present in the cochlea. However, the medical device may also be exposed to moisture (e.g., ethanol, lenium, deionized water, a soap solution, and n-Heptane) during the manufacturing process. Such exposure may initiate the elution of the drug or the dissolution of a resorbable feature carrying the drug, which may leave the medical device carrying an unknown quantity of the drug at the end of the manufacturing process. Additionally, the elution profile of a drug carried by the medical device may be affected at temperatures greater than 150° C., which can be problematic, as temperatures experienced during the manufacture of a cochlear implant, for example, can be greater than 100° C. However, redesigning a cochlear implant manufacturing process to accommodate a drug-delivery feature is not practical or cost-effective.

Embodiments of the present invention provide an independently-manufactured and physically distinct elongate drug-delivery element for an implantable medical device. As such, the drug delivery element can be applied to the medical device after the manufacturing process (e.g., at the time of surgery), which enables the drug-delivery element to be more consistent and reliable. This also enables manufacturing efforts to be focused solely on the successful manufacture of the implantable medical device rather than on manufacturing an integrated assembly of the device and a drug-delivery mechanism.

Additionally, providing an independently-manufactured and physically distinct elongate drug-delivery element to an implantable medical device also increases flexibility in an applied therapy. For example, as described in more detail below, the type, location and/or dosage of a drug applied to a recipient may all be selected after manufacture (e.g., at the time of surgery). Also, in some embodiments of the present invention, an implantable medical device may be manufactured as a universal device which may be complemented with different embodiments of the elongate drug-delivery element. This advantageously enables a single implantable component to be manufactured and inventoried for a length of time not determined by a drug. This is particularly advantageous in those circumstances in which the drug to be delivered via the accessory has a limited shelf life. As another example, the present invention may enable manufacturing efforts to be focused solely on the successful manufacture of the medical device. This may result in reduced manufacturing costs, reduced drug yield loss, reduced handling and contamination of drugs, and other advantages over conventional techniques. Furthermore, the implantable medical device component and the drug-delivery element may be made by different manufacturers. Specifically, an implantable medical device manufacturer may outsource the manufacture of the drug-delivery element to a third party manufacturer that specializes in making such products. Such outsourcing may provide further benefits such as the reduction of the cost of research and development by outsourcing to manufacturers that already have regulatory approval for the desired drugs. This in turn may facilitate the commercialization of a medical device incorporating the drug-delivery element.

As used herein, the term “drug” refers to any bioactive substance now or later developed, including, but not limited to, pharmaceuticals and other chemical compounds such as those intended to provide therapeutic benefits to, or other reactions in, an implant recipient, whether localized or distributed throughout the recipient. Such bioactive substances may include, for example, steroids or other anti-inflammatory drugs to reduce inflammation at the implantation site. Another class of bioactive substances that may be included in the drug-delivery accessories are antibiotics to mitigate bacterial growth related to the implantation of the medical device.

Embodiments of the invention are not necessarily drawn to scale in the accompanying drawings. Rather, the dimensions of elements illustrated in the drawings serve to highlight features of embodiments of the present invention.

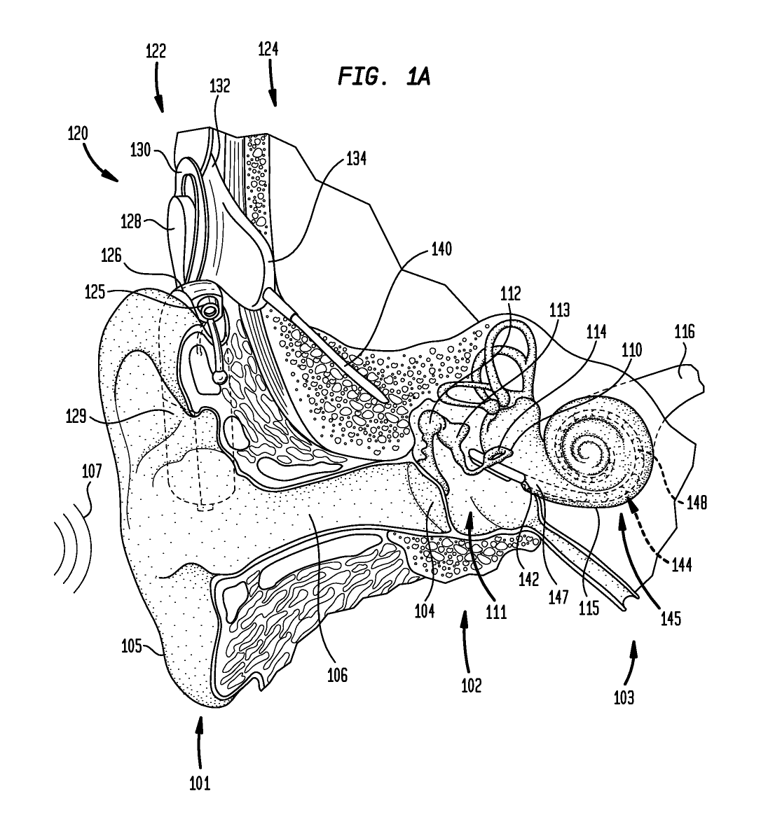

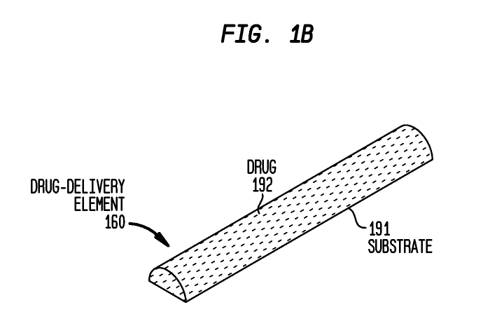

FIG. 1A is a perspective view of an exemplary cochlear implant with which a drug-delivery element in accordance with embodiments of the present invention may be implemented. In fully functional human hearing anatomy, outer ear 101 comprises an auricle 105 and an ear canal 106. A sound wave or acoustic pressure 107 is collected by auricle 105 and channeled into and through ear canal 106. Disposed across the distal end of ear canal 106 is a tympanic membrane 104 which vibrates in response to acoustic wave 107. This vibration is coupled to oval window or fenestra ovalis 110 through three bones of middle ear 102, collectively referred to as the ossicles 111 and comprising the malleus 112, the incus 113 and the stapes 114. Bones 112, 113 and 114 of middle ear 102 serve to filter and amplify acoustic wave 107, causing oval window 110 to articulate, or vibrate. Such vibration sets up waves of fluid motion within cochlea 115. Such fluid motion, in turn, activates tiny hair cells (not shown) that line the inside of cochlea 115. Activation of the hair cells causes appropriate nerve impulses to be transferred through the spiral ganglion cells and auditory nerve 116 to the brain (not shown), where they are perceived as sound. In deaf persons, there is an absence or destruction of the hair cells. A cochlear implant 120 is utilized to directly stimulate the ganglion cells to provide a hearing sensation to the recipient.

FIG. 1A also shows the positioning of cochlear implant 120 relative to outer ear 101, middle ear 102 and inner ear 103. Cochlear implant 120 comprises external component assembly 122 which is directly or indirectly attached to the body of the recipient, and an internal component assembly 124 which is temporarily or permanently implanted in the recipient. External assembly 122 comprises microphone 125 for detecting sound which is outputted to a behind-the-ear (BTE) speech processing unit 126 that generates coded signals which are provided to an external transmitter unit 128, along with power from a power source 129 such as a battery. External transmitter unit 128 comprises an external coil 130 and, preferably, a magnet (not shown) secured directly or indirectly in external coil 130.

Internal component assembly 124 comprises an internal coil housing 132 that receives and transmits power and coded signals received from external assembly 122 to a stimulator unit 134 to apply the coded signal to cochlea 115 via an implanted elongate stimulating assembly 140. An intra-cochlear region 145 of stimulating assembly 140 enters cochlea 115 at cochleostomy region 142. Stimulating assembly 140 comprises a longitudinally aligned and distally extending array 144 of stimulating contacts 148, sometimes referred to as contact array 144 herein, disposed along a length thereof. Although contact array 144 may be disposed on stimulating assembly 140, in most practical applications, contact array 144 is integrated into stimulating assembly 140. As such, for all embodiments of stimulating assembly 140, contact array 144 is generally referred to herein as being disposed in stimulating assembly 140. Stimulating contacts 148 are positioned in stimulating assembly 140 to be substantially aligned with portions of tonotopically-mapped cochlea 115. Signals generated by stimulator unit 134 are typically applied by contact array 144 to cochlea 115, thereby stimulating auditory nerve 116. Additionally, intra-cochlear region 145 of stimulating assembly 140 has a proximal end 147 that is disposed near cochleostomy region 142 when intra-cochlear region 145 is implanted in cochlea 115.

Given the coiling shape of cochlea 115, cochlear implant devices such as stimulating assembly 140 are often constructed using a material, or combination of materials, which curls or is capable of being curled in a manner which follows the curvature of cochlea 115. The portion of stimulating assembly 140 intended to be inserted into cochlea 115 will often have a stiffening stylet (not shown) inserted into a channel, for example a lumen (not shown), which extends distally from the proximate end of stimulating assembly 140, which is not necessarily the same as proximal end 147. During implantation of stimulating assembly 140, the stylet contained in the lumen of stimulating assembly 140 is removed from the proximate end of stimulating assembly 140 as stimulating assembly 140 is inserted into cochlea 115. The act of removing the stiffening stylet from the lumen allows stimulating assembly 140 to curl. In further embodiments of cochlear implant 120, the stiffness of the stylet decreases in response to fluids and/or body temperature allowing stimulating assembly 140 to curl in order to follow the curvature of the inner walls of cochlea 115. In other embodiments of cochlear devices, stimulating assembly 140 is naturally straight without the assistance of a stylet inserted into the lumen. Stimulating assembly 140 is constructed using a flexible material, or is constructed so as to flex upon a fixed amount of force being exerted on the tip or body of stimulating assembly 140 as it is being inserted into cochlea 115. In other embodiments, stimulating assembly 140 has a length which results in it extending to the first turn of cochlea 115. In further embodiments of implanted cochlear devices, the stylet becomes flexible in response to fluids and/or body temperature thereby allowing stimulating assembly 140 to curl so as to follow the curvature of the inner wall of cochlea 115.

As one of ordinary skill in the art will appreciate from the present disclosure, embodiments of the present invention may be advantageously implemented in a variety of implantable medical devices, components, etc. (“devices” herein). Although cochlear implant 120 described above with reference to FIG. 1A is a partially-implantable device, embodiments of the present invention also provide benefits to devices which have limited sources of power such as fully-implantable prosthetic hearing devices including fully-implantable bone-anchored hearing aids, fully-implantable cochlear implants, middle ear implants, and the like. Embodiments of the present invention may also provide benefits to other types of implantable medical devices that have various types of elongate components, such as leads or catheters.

FIG. 1B is a side perspective view of a portion of an exemplary elongate drug-delivery element 160, in accordance with embodiments of the present invention. In embodiments of the present invention, drug-delivery element 160 may have various lengths and may have a different shape than the shape illustrated in FIG. 1B. Drug-delivery element 160 comprises a substrate (or body) 191. A drug 192 is releasably carried in or on (collectively and generally “in” herein) substrate 191 of drug-delivery accessory 194. That is, a drug 192 is releasably secured in substrate 191 such that drug 192 is implanted in the recipient with drug-delivery element 160 so as to complement an implantable device or component (not shown in FIG. 1B). The drug 192 is subsequently released in to the body of the recipient in which the drug-delivery element 160 and its complementary component are implanted. In certain embodiments, the drug 192 carried by substrate 191 may be released via elution or through resorption of substrate 191. In other embodiments, drug 192 can be dispersed in an ionic fluid or solution that is allowed to diffuse or migrate and/or is expelled from pores in substrate 191 under application of a suitable electric field. In such embodiments, substrate 191 may be constructed of a porous metallic material, such as porous platinum.

For ease of illustration, components of drug 192 are schematically illustrated as small line segments distributed throughout substrate 191. It should be appreciated, however, that the quantity of different drugs, the amount of each such drug, the location of such drug or drugs, and so on, may be determined based on the particular substrate 191, drug or drugs 192, the condition or conditions to be treated by the drug or drugs, the implant location, recipient physiology and other factors.

Substrate 191 may be composed of a variety of materials, and have a variety of structures, depending on the particular application and type of drug(s) 192 which substrate 191 is to carry. It should also be appreciated that the mechanism by which drug 192 is releasably secured in substrate 191 of drug-delivery element 160 may be a characteristic of substrate 191, a characteristic of drug 192, or a characteristic of both substrate 191 and drug 192. Additionally or alternatively, an additional treatment or agent may be employed to releasably secure drug 192 in substrate 191.

As discussed elsewhere herein, an elongate drug-delivery element 160 in accordance with embodiments of the present invention may be used to complement a number of different implantable components of a variety of implantable medical devices. For example, referring specifically to cochlear implants, a drug-delivery element in accordance embodiments of the present invention may complement stimulating assemblies, such as stimulating assembly 140 shown in FIG. 1A, for example. For ease of illustration, embodiments of the present invention will be described with reference to an elongate drug-delivery element used in conjunction with a stimulating assembly of a cochlear implant. Such examples are merely illustrative and should not be construed as limiting the present invention. Embodiments of the present invention may also provide benefits to other types of implantable medical devices, and particularly those having any one of various types of implantable elongate components, such as leads or catheters.

It may be desirable for embodiments of the drug-delivery element of the present invention to be constructed of a material that is resorbable, bio-resorbable, bio-degradable, and/or dissolvable (generally and collectively referred to herein as “resorbable”), so that while bioactive substances are being absorbed at the implant site, or after they are absorbed, the drug-delivery element may be partially or completely resorbed by the tissue surrounding the implant site. In certain embodiments, the drug-delivery element is comprised of a resorbable material that partially or completely degrades over time through interaction with various body fluids, through exposure to body temperatures, and/or through interaction with or exposure to any other substance or condition present within a recipient's body.

However, it may also be desirable for the drug-delivery element to be constructed of a non-resorbable material. The use of a non-resorbable material may offer different benefits from the use of a resorbable material, such as the continued provision of spacing or support for other tissue or implanted components. For example, the drug-delivery element may be made of a polymeric material configured to enable bioactive substances to be embedded within the structure of the polymeric material, and to release the bioactive substances either in response to an external catalyst (i.e., a catalyst that is not a substance normally present or a condition normally occurring in a recipient's body) or through the interaction of body fluids and/or body heat that may permeate the accessory.

Embodiments of the drug-delivery element of the present invention may be formed of a semisoft material, such as paste or gel (generally and collectively “paste” herein), that has been combined with one or more drugs and that may be readily applied to an elongate component of an implantable medical device prior to insertion of the elongate component. In alternative embodiments, a drug-delivery element in accordance with embodiments of the present invention may be ultraviolet (UV) cured silicone releasably carrying one or more drugs. In such embodiments, UV curable silicone in an uncured state may be mixed with one or more drugs and then applied to an elongate component of an implantable medical device. Once applied to the elongate component, the silicone may be cured via the application of UV light to form the elongate drug-delivery element mounted on the elongate component.

Additionally, embodiments of the drug-delivery element of the present invention may be manufactured by molding the element from a substance that has been combined with one or more drugs. In certain embodiments, prior to molding, a curable substance in an uncured state is combined with one or more drugs to form a molding mixture. In certain embodiments, the curable substance may be a silicone in its uncured state (e.g., LSR 30). An assembly is then placed into a molding die and injected with the molding mixture containing the one or more drugs. The mixture is then cured by a means suitable for the curable substance used. For example, when room-temperature vulcanization (RTV) silicone is the curable substance, the silicone may be exposed to the appropriate environmental conditions and allowed to cure. Alternatively, when using platinum-cured silicone, the mixture may be cured through appropriate heating, and when ultra-violet (UV) cured silicone is used, the mixture may be cured through exposure to UV light. Subsequently, the completed drug-delivery element may be removed from the die.

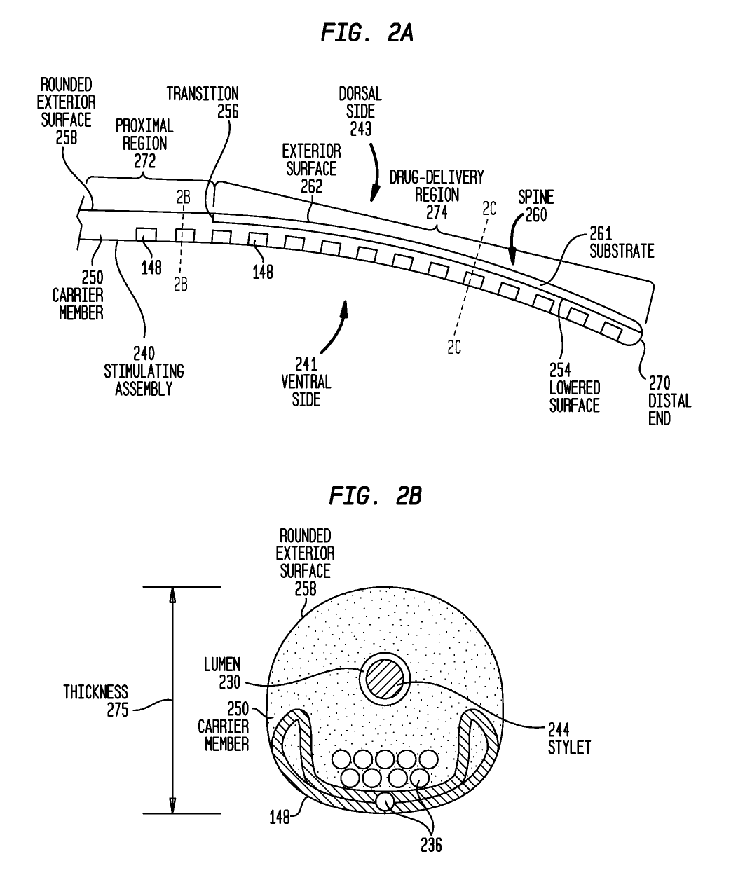

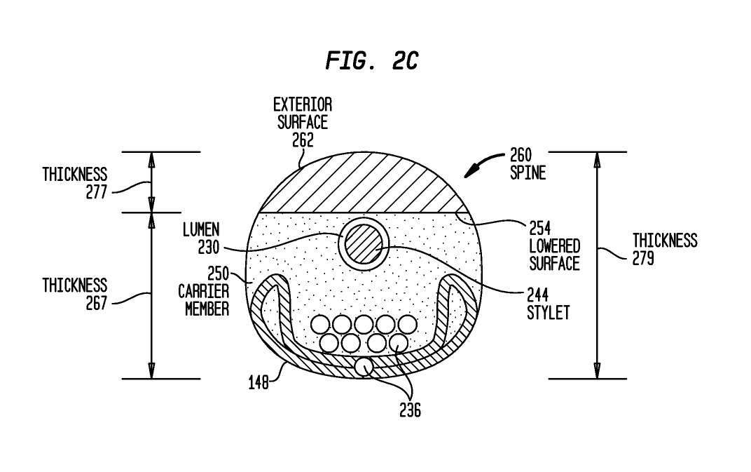

FIG. 2A is a side view of a portion of an implantable medical device having an elongate drug-delivery element mounted on an implantable elongate component of the device, in accordance with embodiments of the present invention. As used herein, an “implantable medical device” includes both partially and fully implantable devices, and includes any elongate drug-delivery element(s) configured to be mounted to the device in accordance with embodiments of the present invention. In the illustrative embodiment of FIG. 2A, the elongate drug-delivery element is an elongate drug-delivery spine 260, and the implantable elongate component is a stimulating assembly 240, which is an embodiment of stimulating assembly 140 of FIG. 1A. Stimulating assembly 240 comprises a carrier member 250 having a ventral side 241 and a dorsal side 243. As illustrated, stimulating assembly 240 comprises a plurality of stimulating contacts 148 extending lengthwise along ventral side 241 of carrier member 250 and disposed in carrier member 250. As used herein, a “ventral side” of a carrier member or stimulating assembly is the side of the carrier member or stimulating assembly configured to face the modiolus when implanted in the cochlea. In some applications, the ventral side of a carrier member or stimulating assembly is the side in which the contact array is disposed. Also, as used herein, a “dorsal side” of a carrier member or elongate stimulating assembly is the side approximately opposite the ventral side of the carrier member or elongate stimulating assembly. It would be appreciated that carrier member 250 may be formed from a number of different materials. In one embodiment, carrier member 250 is formed from a silicone such as a Silastic® material (e.g., polydimehtylsiloxane (PDMS)), while in other embodiments carrier member 250 may be in whole, or in part, a urethane, polyimide, polypropelene, polytetrafluoroethene (PTFE), polyaryletheretherketone, (PEEK) or any other type suitable material.

In the illustrative embodiment of FIG. 2A, carrier member 250 comprises a proximal region 272 and a drug-delivery region 274. FIG. 2B is a cross-sectional view of stimulating assembly 240 taken along section line 2B-2B of FIG. 2A in proximal region 272 of carrier member 250. As illustrated in FIG. 2B, stimulating assembly 240 may comprise a lumen 230 through which a stiffener or stylet 244 may be placed for use in implantation of stimulating assembly 240 in the recipient's cochlea. Additionally, each stimulating contact 148 may be connected to one or more conductive pathways 236 which extend from the stimulating contacts 148 through stimulating assembly 240 to stimulator unit 134 (FIG. 1). In certain embodiments, stimulating assembly 240 comprises 22 stimulating contacts 148, although in other embodiments, stimulating assembly 240 may comprise any number of stimulating contacts. In alternative embodiments, stimulating assembly may have more or fewer conductive pathways 236 than the number shown in FIG. 2B. As illustrated, carrier member 250 has a rounded exterior surface 258 on dorsal side 243 in proximal region 272. Carrier member 250 also has a width 275 between ventral and dorsal sides 241 and 243 in proximal region 272.

As illustrated in FIG. 2A, spine 260 is mounted on dorsal side 243 of carrier member 250 in drug delivery region 274. FIG. 2C is a cross-sectional view of stimulating assembly 240 and elongate drug-delivery spine 260 taken along section line 2C-2C of FIG. 2A in drug-delivery region 274 of carrier member 250. As illustrated in FIGS. 2A and 2C, spine 260 is mounted on a lowered surface 254 of dorsal side 243 of carrier member 250. Unlike rounded exterior surface 258 of proximal region 272, lowered surface 254 of drug-delivery region 274 has a substantially flat cross-section. As illustrated in FIG. 2A, dorsal side 243 of carrier member 250 transitions from rounded exterior surface 258 to lowered surface 254 in an approximately stepwise manner at a transition region 256. In alternative embodiments, carrier member 250 may have a more gradual transition between rounded exterior surface 258 and lowered surface 254. As illustrated, lowered surface 254 extends from transition 256 to distal end 270. In the illustrative embodiments of FIGS. 2A-2C, the thickness 275 of carrier member 250 between ventral and dorsal sides 241 and 243 in proximal region 272 is substantially greater than the thickness 267 of carrier member 250 between ventral side 241 and lowered surface 254 in drug-delivery region 274. In certain embodiments, this difference in thickness is substantially greater than the difference in thickness that results from the typical tapering of a carrier member toward its distal end.

Lowered surface 254 of carrier member 250 provides a surface at which spine 260 may be mounted to carrier member 250 without substantially increasing the overall width of carrier member 250 between ventral and dorsal sides 241 and 243 relative to a typical carrier member not having a lowered surface 254. In the illustrative embodiment of FIGS. 2A-2C, spine 260 has a thickness 277 that is selected such that, when spine 260 is mounted on lowered surface 254, the combined thickness 279 of carrier member 250 in drug-delivery region 274 and spine 260 is substantially the same as the thickness of a typical carrier member at corresponding locations. Exemplary thicknesses 267 and 277 are illustrated in FIG. 2C. In alternative embodiments, thicknesses 267 and 277 may be varied while thickness 279 remains constant. For example, in alternative embodiments having the same total thickness 279, spine 260 may have a greater thickness 277 than illustrated in FIG. 3C while carrier member 250 has a smaller thickness 267 than illustrated in FIG. 3C. Alternatively, in embodiments having the same total thickness 279, spine 260 may have a smaller thickness 277 than illustrated in FIG. 3C while carrier member 250 has a greater thickness 267 than illustrated in FIG. 3C. In other embodiments, carrier member 250 does not include a lowered surface 254. In such embodiments, spine 260 is mounted to dorsal side 243 of a carrier member 250 that does not include a lowered surface 254.

As shown, an exterior surface 262 of spine 260 is substantially flush with rounded exterior surface 258 at transition 256. In the illustrative embodiment of FIGS. 2A-2C, exterior surface 262 is rounded. In alternative embodiments, exterior surface 258 may have other shapes and such exterior surfaces 258 may not fit flush with outer surfaces of carrier member 250. In certain embodiments, stimulating assembly 240 gradually tapers toward distal end 270. In such embodiments, one or both of spine 260 and carrier member 250 in drug-delivery region may be tapered.

In some embodiments of the present invention, spine 260 may be formed of a solid material. In such embodiments, spine 260 may be manufactured by molding the element from a substance that has been combined with one or more drugs, as described above. Spine 260 may be molded such that it is dimensioned to fit on lowered surface 254 so as to form a contiguous component surface with outer surfaces of stimulating assembly 240. That is, spine 260 may be dimensioned such that outer surfaces of spine 260 fit flush with outer surfaces of stimulating assembly 240.

In embodiments in which spine 260 is a solid, spine 260 may be bonded to carrier member 250 in order to mount spine 260 on carrier member 250. In one embodiment, the bonding is performed by disposing a glue layer on one or more of lowered surface 254 and spine 260 and pressing together lowered surface 254 and spine 260 prior to implantation. This may be performed manually or with a simple press-tool that aligns the two components and presses them together with a predefined amount of pressure. Alternatively, a liquid glue may be applied between lowered surface 254 and spine 260. In one preferred embodiment, the liquid glue sets and/or cures rapidly. In another embodiment, a UV-cured glue is pre-applied to lowered surface 254 and/or spine 260, or is applied as a liquid, or is a separate component that is inserted between lowered surface 254 and spine 260. In one embodiment, a liquid perfluoropol polymer such as that described in International Application WO 2007/021620 A2 may be utilized. Other adhesives include, but are not limited to, fibrin glues, cyanoacrylates, polyurethane adhesives, silicone adhesives, and UC-cured acrylics. In another embodiment, chemical surface modification may be utilized to attain a desired bonding. For example, in one embodiment, covalently bonded proteins, or sulfonation may be performed to increase the wettability of the surface.

In other embodiments of the present invention, spine 260 may be constructed of a semisoft material, such as a paste. In such embodiments, a paste releasably carrying one or more drugs is applied to lowered surface 254 to form drug-delivery spine 260. The paste may be applied to lowered surface 254, for example, via a syringe carrying the paste. The paste may be manufactured such that it releasably carries one or more drugs, or a suitable carrier paste may be combined with one or more drugs before applying the paste to carrier member 250. In some embodiments, the paste is not a readily curable substance. In alternative embodiments, the paste is readily curable. For example, in certain embodiments, UV cured silicone carrying one or more drugs may be applied to carrier member 250 prior to implantation, as described above. In such embodiments, UV curable silicone in an uncured state may be mixed with one or more drugs and then applied to lowered surface 254 of carrier member 254. Once applied to carrier member 250, the silicone may be cured via the application of UV light to form spine 260 mounted on carrier member 250. Then, after implantation of carrier member 250, the one or more drugs may elute from the porous silicone formed via the UV curing process.

In certain embodiments, drug-delivery spine 260 may be constructed of a non-resorbable material. A non-resorbable spine 260 will remain on carrier member 250 after implantation and the release of drug(s) carried by spine 260. In alternative embodiments, drug-delivery spine 260 may be constructed of a resorbable material. For example, a substrate 261 of spine 260 may be constructed of a resorbable material. In such embodiments, resorbable spine 260 will not remain in the cochlea indefinitely. By contrast, a spine 260 having a substrate 261 fabricated from silicone, for example, would remain in the cochlea after releasing drug(s). By remaining in the cochlea, spine 260 could create a location for harmful microbes to gather since spine 260 is separate from carrier member 250. In certain embodiments, resorbable drug-delivery elements are constructed of one or more biodegradable polymers. Examples of suitable biodegradable polymers include poly(acrylic acid), poly(ethylene glycol), poly(vinylpyrrolidone), poly(hydroxybutyrate), poly(lactide-co-glycolide), and polyanhydrides. In some embodiments, it is desirable to achieve a sustained drug release over a period of up to ninety days, for example. In certain embodiments, a resorbable spine will have an initial release of the drug upon implantation, followed by a second phase of additional drug release that is sustained over a longer period of time. Additionally, the longer the sustained drug release period lasts (preferably up to a maximum of ninety days), the more benefit a recipient may receive from the drug release.

Drug-delivery spines described herein in accordance with embodiments of the present invention may be manufactured separately from, for example, a carrier member of an implantable medical device, and may be mounted on the carrier member subsequent to the carrier member's manufacture or sterilization. Providing independently-manufactured and physically distinct (i.e., “separate”) drug-delivery spines in accordance with embodiments of the present invention increases flexibility for the application of therapy. In certain embodiments, separate drug-delivery spines releasably carrying different types of drugs are provided, allowing the type of drug to be applied to be selected after manufacture of the implantable device, such as at the time of surgery. Accordingly, when the stimulating assembly is to be inserted through a cochleostomy, for example, a drug that encourages fibrous tissue growth to achieve a faster and stronger cochleostomy seal may be selected. Alternatively, when the stimulating assembly is to be inserted through the round window, a drug that encourages sealing of the round window may be selected. This drug may be different from the drug that encourages fibrous tissue growth since no new bone growth is necessary after inserting an electrode assembly through the round window. In addition, multiple drug-delivery spines releasably carrying different drugs may be selected so that multiple different drugs can be applied to a recipient simultaneously.

In certain embodiments of the present invention, separate drug-delivery spines having different dosages may be provided, allowing the dosage of the drug(s) to be selected after manufacture of the implantable device. For example, drug-delivery spines releasably carrying different amounts of a drug may be provided. Additionally or alternatively, the dosage of the drug(s) may be selected by choosing the number of drug-delivery spines to position on the carrier member. For example, a larger number of drug-delivery spines, each occupying a portion of lowered surface 254, may be positioned on the carrier member to apply a larger dose of a drug, and a smaller number of drug-delivery spines, each occupying a portion of lowered surface 254, may be positioned on the carrier member to apply a smaller dose of a drug. In embodiments in which the drug-delivery spine is applied to the carrier member as a paste, the dosage of the drug may be selected by applying more or less paste to the carrier member.

Additionally, a chosen location within a recipient may be targeted by applying a drug-delivery paste as spine 260 to a selected location on carrier member 250 configured to be adjacent to the chosen location when implanted. Alternatively, the chosen location may be targeted by applying a solid spine 260 having a length smaller than the length of lowered surface 254 to a selected location along lowered surface 254. In certain embodiments, the location chosen for application of the drug may be a location that is advantageous for the release of the drug. One such location is adjacent to the cochlear aqueduct. The cochlear aqueduct is connected to a port in the cochlea, and there is therefore more movement of cochlear fluid (e.g., back-and-forth movement) adjacent to the cochlear aqueduct than in other places of the cochlea. Another location that may be advantageous for the release of the drug is a location adjacent to the stapes footplate. In many cochlear implant recipients, the stapes footplate still moves, creating pressure waves within the cochlea. Thus, drugs may travel well from a location in the cochlea that is adjacent to the stapes footplate. Additionally, various combinations of the type, location and/or dosage of one or more drugs may be selected in accordance with embodiments of the present invention.

FIG. 3A is a top view of a portion of an implantable medical device having an elongate drug-delivery element mounted on an implantable elongate component of the device, in accordance with embodiments of the present invention. In the illustrative embodiment of FIG. 3A, the elongate drug-delivery element is an elongate drug-delivery spine 360, and the implantable elongate component is a stimulating assembly 340, which is an embodiment of stimulating assembly 140 of FIG. 1A. Stimulating assembly 340 is similar to stimulating assembly 240, except that, instead of a lowered surface 254, carrier member 250 of stimulating assembly 340 comprises an elongate recess 354 in dorsal side 243 in which an elongate drug-delivery spine 360 may be mounted. As illustrated in FIG. 3A, elongate recess 354 that extends along carrier member 250 such that a longitudinal axis of elongate recess 354 is substantially parallel with a longitudinal axis of carrier member 250. In the illustrative embodiment of FIG. 3A, recess 354 extends from a location adjacent distal end 270 toward a proximal end (e.g., proximal end 147 of FIG. 1A) of carrier member 250. As illustrated in FIG. 3A, recess 354 does not extend to distal end 270. In alternative embodiments, recess 354 may extend to distal end 270 such that a spine 360 mounted therein forms a portion of distal end 270. In still other embodiments, recess 354 may extend to distal end 270, where a distal boundary of recess 354 is formed.

FIG. 3B is a cross-sectional view of stimulating assembly 340 and elongate drug delivery spine 360 taken along section line 3B-3B of FIG. 3A, in accordance with embodiments of the present invention. In the illustrative embodiment of FIG. 3B, spine 360 is mounted in recess 354, which has a rounded cross-sectional shape. Spine 360 has a rounded cross-sectional shape that is complementary to the shape of recess 354. In certain embodiments, spine 360 may be formed of a solid material, as described above in relation to spine 260 of FIGS. 2A-2C. A solid spine 360 may be dimensioned so as to fit in recess 354. In certain embodiments, spine 360 is dimensioned to fit loosely in recess 354. In such embodiments, spine 360 may be bonded within recess 354, as described above in relation to FIGS. 2A-2C.

In other embodiments, spine 360 may be configured to be mechanically locked into recess 354. In certain embodiments, spine 360 is has a width that is slightly larger than the width of recess 354 so that spine 360 will form an interference fit with recess 354 when inserted (e.g., pressed) into recess 354. In such embodiments, spine 360 may be mechanically locked into recess 354, so bonding spine 360 inside recess 354 is unnecessary. Additionally, because silicone is flexible, carrier member 250 can accommodate the insertion of spine 360 when carrier member 250 is constructed of silicone. As used herein, a first element is “mechanically locked” a second element when the second element securely retains the first element via the interaction of the shapes of the elements. Thus, a first element secured in or to a second element via any type of adhesive or bonding is not considered to be “mechanically locked” in or to the second element, as used herein. In addition, spine 360 may either partially or completely fill recess 354. For example, spine 360 may fill the entire length of recess 354, or fill only a portion of the length.

As described above, drug-delivery elements in accordance with embodiments of the present invention may be constructed of a resorbable material. For example, spine 360 may be constructed of a solid, resorbable material. In embodiments in which the drug-delivery spine is solid and resorbable, the drug delivery spine may provide advantages for the insertion of the stimulating assembly into a recipient's cochlea. For example, many cochlear implant stimulating assemblies are configured with intra-cochlear regions that are pre-curved to substantially match the shape of the modiolus of the cochlea such that, after insertion into the cochlea, the intra-cochlear region will substantially conform to the modiolus when it assumes its pre-curved configuration. In embodiments in which the drug-delivery spine is solid and resorbable, the drug-delivery spine may be is configured to bias the elongate component into a less-curved configuration in which the elongate component is less curved (i.e., has a greater radius of curvature) than in the pre-curved (i.e., fully curved) configuration. Thus, the drug-delivery element may be used to assist in holding the straight the pre-curved intra-cochlear region to facilitate insertion. Once inserted in the cochlea, drug-delivery spine will gradually be resorbed thus allowing the intra-cochlear region to fully assume its pre-curved configuration. Because it is desirable to achieve a sustained drug release over a period of up to ninety days, for example, a solid drug-delivery spine configured to resorb over a similar period may be provided. In such embodiments, the intra-cochlear region of the stimulating assembly may not achieve its pre-curved configuration hugging the modiolus until the end of that period. As such, a visit to the audiologist for an initial or additional fitting session should be scheduled to coincide with the complete resorption of the drug-delivery spine and the resulting final positioning of the intra-cochlear region.

Alternatively, drug-delivery elements in accordance with embodiments of the present invention may be constructed of a non-resorbable material, as described above. For example, spine 360 may be constructed of a solid, non-resorbable material. In embodiments in which the drug-delivery spine is solid and non-resorbable, the drug delivery spine may assist in holding the intra-cochlear region such that it conforms more closely to the modiolus. It has been found that in order for the electrode contacts of a cochlear implant stimulating assembly to be effective, the magnitude of the currents flowing from these electrode contacts and the intensity of the corresponding electric fields, are a function of the distance between the electrode contacts and the modiolus. If this distance is relatively great, the threshold current magnitude must be larger than if the distance is relatively small. Moreover, the current from each electrode contact may flow in all directions, and the electrical fields corresponding to adjacent electrode contacts may overlap, thereby causing cross-electrode interference. In order to reduce the threshold stimulation amplitude and to eliminate cross-electrode interference, it is advisable to keep the distance between the electrode array and the modiolus as small as possible. This is best accomplished by providing the electrode array in the shape which generally follows the shape of the modiolus. Also, this way the delivery of the electrical stimulation to the auditory nerve is most effective as the electrode contacts are as close to the auditory nerves that are particularly responsive to selected pitches of sound waves.

As described above, many cochlear implant stimulating assemblies are configured with intra-cochlear regions that are pre-curved to substantially match the modiolus of the cochlea such that, after insertion into the cochlea, the intra-cochlear region will substantially match the shape of the modiolus when it assumes its pre-curved configuration. In embodiments in which the drug-delivery spine is solid and non-resorbable, the drug-delivery spine may also be pre-curved like the intra-cochlear region. In such embodiments, the pre-curved drug-delivery spine may be mounted to the intra-cochlear region in its pre-curved configuration. Then, when the intra-cochlear region is straightened for insertion, the drug-delivery spine is straightened along with the intra-cochlear region. For example, a such as a straightening stylet may be used to bias both the intra-cochlear region and the drug-delivery spine into a substantially straight configuration for insertion. After the intra-cochlear region is advanced off of the straightening element and into the cochlea, the intra-cochlear region assumes its pre-curved configuration substantially matching the shape of the modiolus. At this time, the pre-curved drug-delivery spine may also bias the intra-cochlear region to conform more closely to the modiolus. Additionally, because the drug-delivery spine is non-resorbable, the spine will continually hold the intra-cochlear region in place conforming to the modiolus.

In alternative embodiments, spine 360 may be constructed of a semisoft material, such as a paste, as described above in relation to spine 260 of FIGS. 2A-2C. In such embodiments, recess 354 is partially or completely filled with a paste releasably carrying one or more drugs to form drug-delivery spine 360. Recess 354 may be filled with the paste, for example, via a syringe carrying the paste. In embodiments of the present invention, spine 360 may be resorbable or non-resorbable, as described above. Additionally, in accordance with embodiments of the present invention, spine 360 and recess 354 may have various cross-sectional shapes other than those illustrated in FIG. 3B.

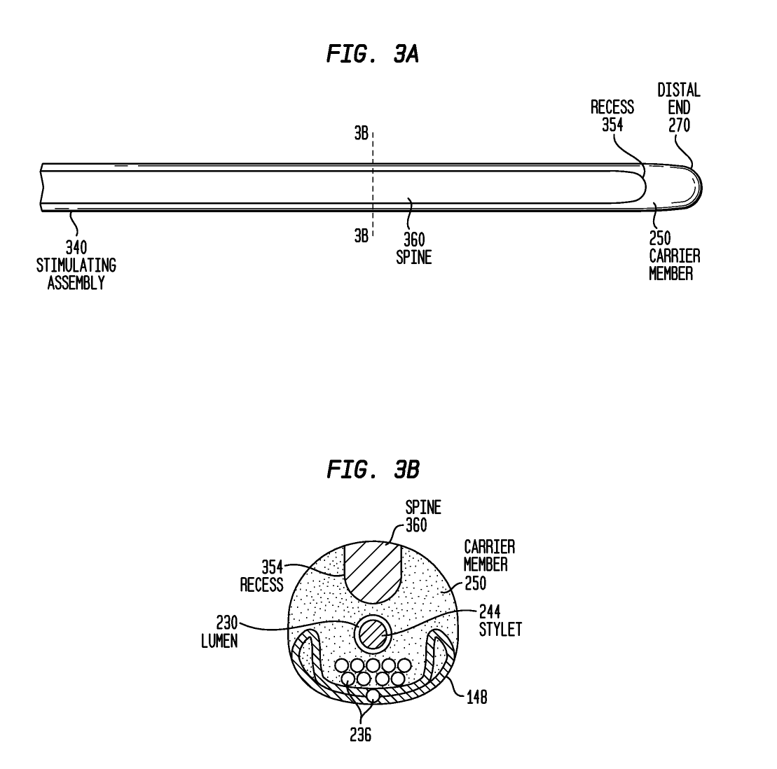

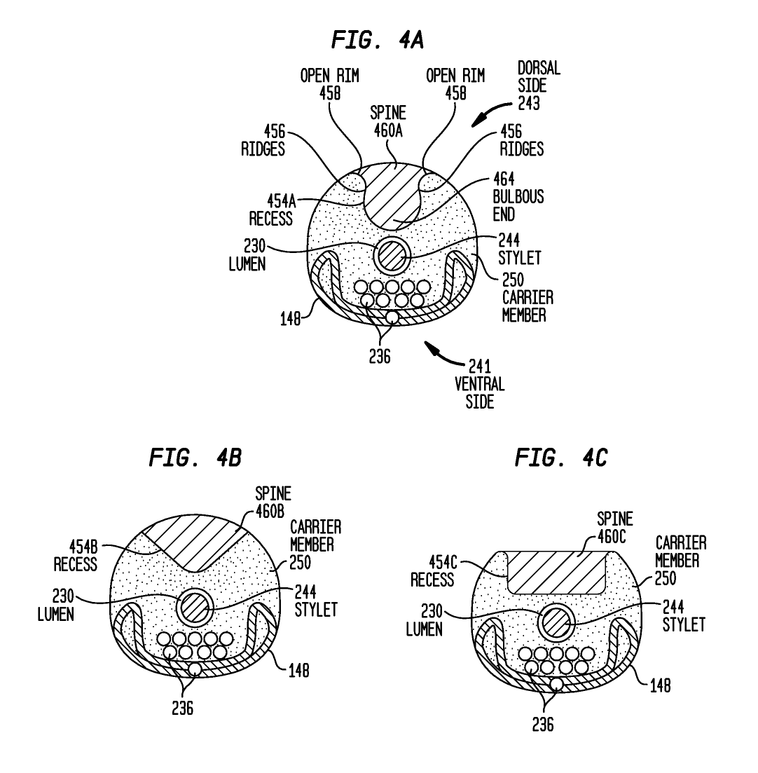

FIGS. 4A-4C are cross-sectional views of alternate embodiments of stimulating assembly 340 and elongate drug-delivery spine 360 of FIGS. 3A and 3B, in accordance with embodiments of the present invention. FIG. 4A illustrates an elongate drug-delivery spine 460A and a recess 454A, which are alternate embodiments of spine 360 and recess 354, respectively. As shown in FIG. 4A, recess 454C has a concave, bulbous shape. Spine 460A has a convex, bulbous shape that corresponds to the shape of recess 454A such that spine 460A fits into recess 454A. Like spine 360, spine 460A may be constructed of a solid material or a semisoft material, such as a paste. When spine 460A is constructed of a solid material, spine 460A may be a solid rod such as may be mechanically locked in recess 454A. As illustrated in FIG. 4A, bulbous recess 454A includes ridges 456, and spine 460A includes a bulbous end 464. In certain embodiments, the width of bulbous end 464 is greater than the distance between ridges 456. In such embodiments, once bulbous end 464 is pressed through ridges 456, through ridges 456 substantially prevent bulbous end 464 from passing through ridges 456 and out of recess 454A. As such, spine 460A may be held in place in recess 454A by ridges 456. Additionally, recess 454A includes open outer rims 458 on either side of recess 454A. Open outer rims 458 provide are more advantageous than outer rims that form an overhang over the interior of recess 454A. Such overhangs could create one or more locations for harmful microbes to gather. However, open outer rims 458, which do not hang over recess 454A, do not provide such locations for microbes to gather. Additionally, as described above in relation to spine 260, in alternate embodiments, recess 454A may be filled with a paste releasably carrying one or more drugs in order to form spine 460A, or may be filled with UV curable silicone mixed with one or more drugs and subsequently cured to form spine 460A. In the illustrative embodiment of FIG. 4A, an upper surface of spine 460A fits flush with an outer surface of carrier member 250 when mounted in recess 454A. In alternative embodiments, spine 460A may not fit flush. Seems to referring to circular rod in FIG. 4a—add—for example, circular spine such as can be housed in FIG. 4a, etc.

FIG. 4B illustrates an elongate drug-delivery spine 460B and a recess 454B, which are alternate embodiments of spine 360 and recess 354, respectively. As shown in FIG. 4B, spine 460B and recess 454B each have a wedge-shaped cross-section, and spine 460B is dimensioned to fit in recess 454B. Like spine 360, spine 460B may be constructed of a solid material or a semisoft material, such as a paste. As described above with regard to spine 260, when spine 460B is constructed of a solid material, spine 460B may be mounted in recess 454B by bonding spine 460B inside of recess 454B. In alternate embodiments, recess 454B may be filled with a paste releasably carrying one or more drugs to form spine 460B, or may be filled with UV curable silicone mixed with one or more drugs and subsequently cured to form spine 460B, as described above in relation to spine 260. In the illustrative embodiment of FIG. 4B, an upper surface of spine 460B fits flush with an outer surface of carrier member 250 when mounted in recess 454B. In alternative embodiments, spine 460B may not fit flush.

FIG. 4C illustrates an elongate drug-delivery spine 460C and a recess 454C, which are alternate embodiments of spine 360 and recess 354, respectively. As shown in FIG. 4C, spine 460C and recess 454C each have a rectangular cross-section, and spine 460C is dimensioned to fit in recess 454C. Like spine 360, spine 460C may be constructed of a solid material or a semisoft material, such as a paste. In certain embodiments in which spine 460C is constructed of a solid material, spine 460C may have a width slightly larger than the width of recess 454C. In such embodiments, spine 460C may be mechanically locked into recess 454C via an interference fit. Accordingly, bonding spine 460C inside recess 454C is not necessary, but may be performed to further secure spine 460C in recess 454C if desired. In alternate embodiments, recess 454C may be filled with a paste releasably carrying one or more drugs to form spine 460C, or may be filled with UV curable silicone mixed with one or more drugs and subsequently cured to form spine 460C, as described above in relation to spine 260. In the illustrative embodiment of FIG. 4C, an upper surface of spine 460C fits flush with an outer surface of carrier member 250 when mounted in recess 454C. In alternative embodiments, spine 460C may not fit flush.

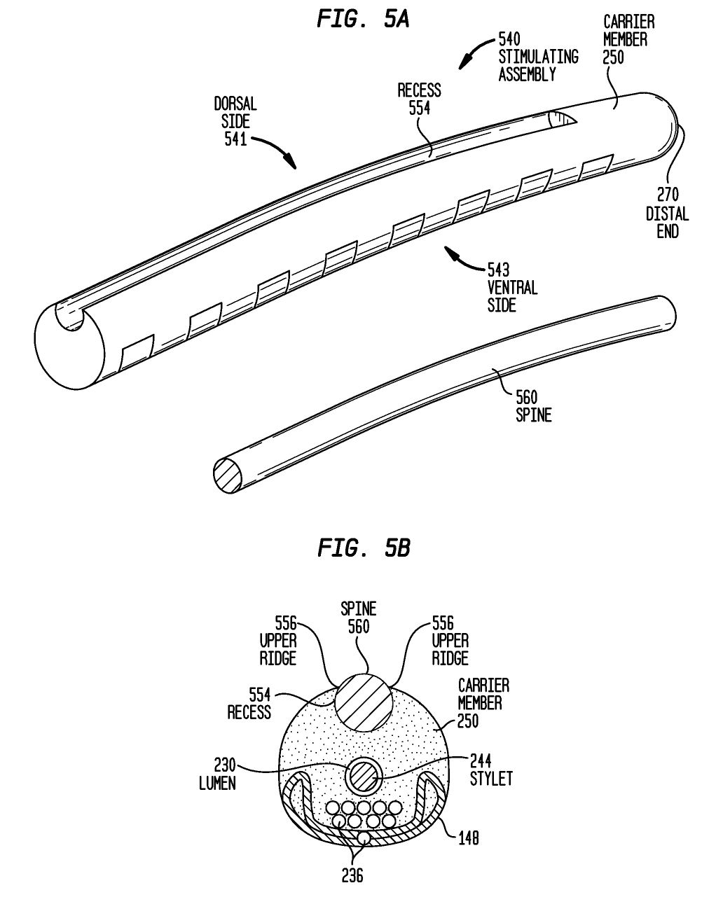

FIG. 5A is a side perspective view of a portion of an implantable medical device having an elongate drug-delivery element that mechanically locks into a recess in an implantable elongate component of the device, in accordance with embodiments of the present invention. In the illustrative embodiment of FIG. 5A, the elongate drug-delivery element is an elongate drug-delivery spine 560, and the implantable elongate component is a stimulating assembly 540, which is an embodiment of stimulating assembly 140 of FIG. 1A. FIG. 5B is a cross-sectional view of stimulating assembly 540 when spine 560 is disposed in recess 554. Stimulating assembly 540 is similar to stimulating assembly 340, except that recess 554 of stimulating assembly 540 has a semicircular cross-sectional shape. In the illustrative embodiment of FIG. 5A, spine 560 is formed of a solid material, as described about in relation to spine 260, and is shaped like a rod having a circular cross-section. Spine 560 is configured to be mounted to stimulating assembly 540 by mechanically locking into recess 554.

In certain embodiments, the diameter of the cross-section of spine 560 is greater than the distance between upper ridges 556 of recess 554. In such embodiments, spine 560 may be positioned in recess 554 by pressing the majority of spine 560 through upper ridges 556. Once the majority of spine 560 has been pressed through upper ridges 556, the center of spine 560, where spine 560 has its greatest width, may not readily pass through upper ridges 556 to exit recess 554. Accordingly, spine 560 may be mechanically locked into recess 554. Unlike the embodiments illustrated in FIGS. 4A-4C, when inserted into recess 554, the upper surface of spine 560 does not fit flush with the outer surface of carrier member 250, but instead protrudes from the surface of carrier member 250. Additionally, carrier member 250 of stimulating assembly 540 may have more stability than carrier member 250 of the embodiments of FIGS. 4a-4C because recess 554 is not as deep or wide as recesses 454A, 454B and 454C.

Spine 560 is similar to embodiments of spine 260 formed of a solid material, as described above. In certain embodiments, spine 560 is dimensioned to substantially fill the length of recess 554. In other embodiments, spine 560 fills only a portion of the length of recess 554 when mounted therein. In such embodiments, multiple spines 560, each only partially filling recess 554, may be mounted in recess 554. When multiple spines 560 are mounted in recess 554, the spines may be substantially similar. In other embodiments, the multiple spines may differ in one or more characteristics, such as the type of drug(s) releasably carried, the dosage of the drug(s), length, etc.

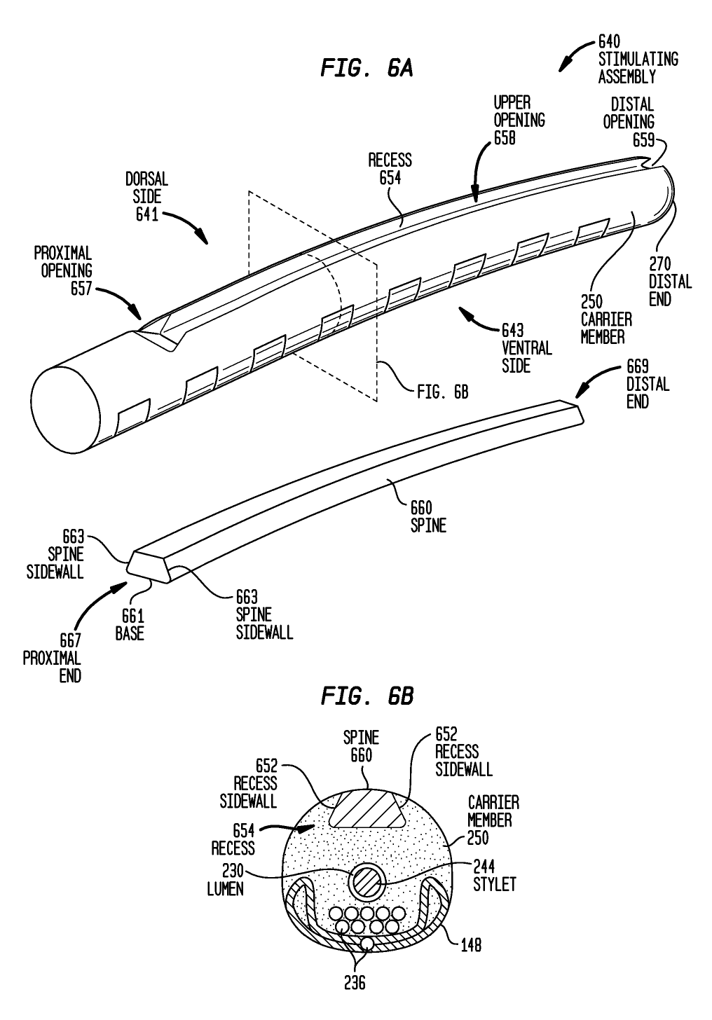

FIG. 6A is a side perspective view of a portion of an implantable medical device having an implantable elongate component and an elongate drug-delivery element that may be mounted on an implantable elongate component of the device, in accordance with embodiments of the present invention. In the illustrative embodiment of FIG. 6A, the elongate drug-delivery element is an elongate drug-delivery spine 660, and the implantable elongate component is a stimulating assembly 640, which is an embodiment of stimulating assembly 140 of FIG. 1A. FIG. 6B is a cross-sectional view of stimulating assembly 640 along the plane illustrated in FIG. 6A when spine 660 is disposed in recess 654. Stimulating assembly 640 is similar to stimulating assembly 340, except that recess 654 of stimulating assembly 640 has a trapezoidal cross-sectional shape, a proximal opening 657 and a distal opening 659 at distal end 270. In certain embodiments, spine 660 forms a portion of distal end 270 at distal opening 658 when mounted on carrier member 250 of stimulating assembly 640. In the illustrative embodiment of FIG. 6A, spine 660 is solid and is shaped like a rod having a trapezoidal cross-section. As shown in FIG. 6B, spine 660 has a substantially trapezoidal cross-section and is dimensioned so that it may be inserted into correspondingly-shaped trapezoidal recess 654. In the illustrative embodiment of FIGS. 6A and 6B, the dimensions of recess 654 prevent spine 660 from being inserted or removed through upper opening 658 of recess 654. Because the base 661 of spine 660 is wider than upper opening 658 of recess 654, recess sidewalls 652 abut spine sidewalls 663, and thereby prevent spine 660 from being removed through upper opening 658.

In the illustrative embodiment of FIGS. 6A and 6B, spine 660 may be inserted into recess 654 by inserting proximal end 667 of spine 660 into distal opening 659 of recess 654 and then sliding spine 660 into recess 654 until proximal end 667 is located near proximal opening 657 of recess 654. Alternatively, spine 660 may be inserted into recess 654 by inserting distal end 669 of spine 660 into proximal opening 657 of recess 654 and then sliding the remainder or spine 660 through proximal opening 657 and into recess 654 until distal end 669 is located near distal opening 659 of recess 654. In the illustrative embodiment of FIGS. 6A and 6B, recess 654 comprises both a proximal opening 657 and a distal opening 659. In alternative embodiments, recess 654 may comprise only one of proximal and distal openings 657 and 659 through which spine 660 may be inserted into recess 654. Additionally, in certain embodiments, spine 660 may be slightly larger than recess 654 so that spine 660 will form an interference fit with recess 654 when inserted (e.g., pressed) into recess 654. Accordingly, bonding spine 660 inside recess 654 is not necessary, but may be performed to further secure spine 660 within recess 654.

Spine 660 is similar to embodiments of spine 260 formed of a solid material, as described above. In certain embodiments, spine 660 is dimensioned to substantially fill the length of recess 654. In other embodiments, spine 660 fill only part of the length of recess 654 when mounted therein. In such embodiments, multiple spines 660, each only partially filling recess 654, may be mounted in recess 654. When multiple spines 660 are mounted in recess 654, the spines may be substantially similar. In other embodiments, the multiple spines may differ in one or more characteristics, such as the type of drug(s) releasably carried, the dosage of the drug(s), length, etc.

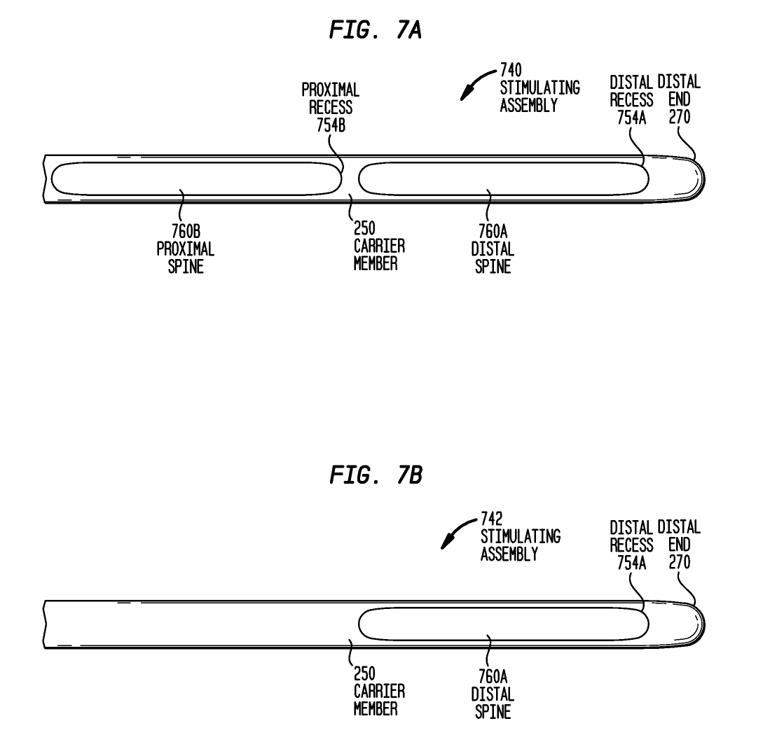

FIG. 7A is a top view of a portion of an implantable medical device having a plurality of elongate drug-delivery elements mounted on an implantable elongate component of the device, in accordance with embodiments of the present invention. In the illustrative embodiment of FIG. 7A, the elongate drug-delivery elements are elongate drug-delivery spines 760A and 760B, and the implantable elongate component is a stimulating assembly 740, which is an embodiment of stimulating assembly 140 of FIG. 1A. Stimulating assembly 740 is similar to stimulating assembly 340, except that, instead of a single recess 354, carrier member 250 of stimulating assembly 740 comprises multiple recesses, namely a distal recess 754A and a proximal recess 754B. Distal and proximal recesses 754A and 754B are arranged in stimulating assembly 704 such that distal recess 754A is disposed nearer to distal end 270, while proximal recess 754B is disposed nearer to proximal end 147 of intra-cochlear region 145 of stimulating assembly 740 (see FIG. 1A). In certain embodiments, distal and proximal recesses 754A and 754B may each have a length that is substantially smaller than the length of recess 354 (see FIG. 3A). Additionally, in embodiments of the present invention, distal and proximal recesses 754A and 754B may have different lengths than those illustrated in FIG. 7A. For example, distal and proximal recesses 754A and 754B may each be shorter or longer than illustrated in FIG. 7A, may have different lengths, and may be separated by a greater distance along carrier member 250.

In the illustrative embodiment of FIG. 7A, a distal spine 760A is mounted in distal recess 754A and a proximal spine 760B is mounted in proximal recess 754B. Distal spine and recess 760A and 754A may have a configuration similar to any of the configurations described herein in relation to embodiments of the present invention. Proximal spine and recess 760B and 754B may also have a configuration similar to any of the configurations described herein in relation to embodiments of the present invention. Additionally, distal spine and recess 760A and 754A may have a different configuration than proximal spine and recess 760B and 754B. Distal and proximal spines 760A and 760B may each be formed of any of the materials described herein in accordance with embodiments of the present invention. In some embodiments, distal and proximal spines 760A and 760B are substantially similar.

In other embodiments, distal and proximal spines 760A and 760B may differ in one or more characteristics, such as the type of drug(s) releasably carried, the dosage of the drug(s), length, etc. As such, stimulating assembly 740 allows for different types of drugs to be applied to different locations within a recipient, when distal and proximal spines 760A and 760B releasably carry different drugs. Such application of multiple drugs in a recipient can be beneficial in the context of a cochlear implant, for example. In one specific example, referring to FIG. 1A, after implanting stimulating assembly 140 into cochlea 115 through cochleostomy region 142, sealing the tissue at cochleostomy region 142 is important so that cochleostomy region 142 does not become a pathway for pathogens. Additionally, it has been theorized that a delay in forming a cochleostomy seal can reduce residual hearing. As such, it is desirable to apply a drug, such as ciprofloxacin, which encourages rapid and strong formation of a cochleostomy seal at or near cochleostomy region 142. However, application of this type of drug is not as desirable in more apical regions of the cochlea, where fibrous tissue growth impedes stimulation. Rather, the preferred drug would maintain the spiral ganglion cells.

Also, in recipients with residual hearing, the residual hearing is typically in a low frequency range. As such, for these recipients, a preferred drug would preserve function in the region of the cochlea mapped to that low frequency range, and in a region of the cochlea mapped to a high frequency range, the preferred drug would maintain the spiral ganglion cells. Accordingly, an anti-inflammatory drug, such as dexamethasone, may be beneficial in more apical regions of the cochlea. Such an anti-inflammatory drug may assist in preserving residual hearing in regions of the cochlea mapped to relatively low frequencies, and assist in maintaining the spiral ganglion cell in regions of the cochlea mapped to relatively high frequencies. However, the anti-inflammatory drug must be kept a sufficient distance away from the cochleostomy site (preferably no closer than 3.5 mm from the cochleostomy site) so that the drug will not interfere with the healing of an insertion site (e.g., a cochleostomy or the round window). For example, in certain embodiments, distal recess 754A may be formed in carrier member 250 such that distal recess 754A will be located no closer than 3.5 mm from the cochleostomy site upon insertion of carrier member 250. As such, any drug disposed in distal recess 754A may be located no closer than 3.5 mm from the cochleostomy site. In other embodiments, all recesses in carrier member, including proximal recess 754B, may be formed in carrier member 250 such that no recess will be located closer than 3.5 mm from the cochleostomy site upon insertion of carrier member 250.

In certain embodiments of the present invention, a first type of drug may be selected for application at a first location, and a second type of drug may be selected for application at a second location. For example, in some embodiments, distal and proximal recesses 754A and 754B may be located in carrier member 250 such that, after implantation of carrier member 250, proximal recess 754B is located adjacent to cochleostomy region 142 and distal recess 754A is located in a more apical region of the cochlea. In such embodiments, a proximal spine 760B may releasably carry a drug that is beneficial for the formation of the cochleostomy seal while a distal spine 760A may releasably carry a drug that is beneficial for a more apical region of the cochlea. As an example, a proximal spine 760B releasably carrying ciprofloxacin may be mounted in a proximal recess 754B prior to insertion, and a distal spine 760A releasably carrying dexamethasone, for example, may be mounted in a distal recess 754A prior to insertion. As such, after implantation of carrier member 250, proximal spine 760B carrying ciprofloxacin is located adjacent to cochleostomy region 142 and distal spine 760A carrying dexamethasone is located in a more apical region of the cochlea.

FIG. 7B is a top view of a portion of an implantable medical device having an elongate drug-delivery element mounted on an implantable elongate component of the device, in accordance with embodiments of the present invention. In the illustrative embodiment of FIG. 7B, the elongate drug-delivery element is an elongate drug-delivery spine 760A and the implantable elongate component is a stimulating assembly 742, which is an embodiment of stimulating assembly 140 of FIG. 1A. Stimulating assembly 742 is similar to stimulating assembly 740, except that carrier member 250 of stimulating assembly 742 comprises only distal recess 754A and distal spine 760A, and not a proximal recess 754B and proximal spine 760B. In certain embodiments, distal recess 754A is confined to a location near distal end 270. In such embodiments, stimulating assembly 742 may target only an apical region of the cochlea for drug delivery via distal spine 760A. As such, stimulating assembly may be used to deliver a drug beneficial at an apical region of the cochlea but not beneficial to a cochleostomy region 142.

The types of drugs that may releasably carried by elongate drug-delivery elements in accordance with embodiments of the present invention include anti-inflammatories (e.g., dexamethasone, cortisol, prednisolone, triamcinolone), corticosteroids (e.g., dexamethasone, bethamethasone, clobethasole, diflorasone, fluocinolone, triamcinolone, salt ester), biological factors (e.g., angiogenic factors, neurotrophic factors), neurotrophic factors (e.g., nerve growth factor (NGF), fibroblast growth factor (FGF), brain-derived neurotrophic factor (BDNF), glial cell-derived neurotrophic factor (GDNF), neurotrophin-3 (NT-3), neurotrophin-4 (NT-4), leukaemia inhibitory factor (LIF), transforming growth factor (TGF)), antimicrobials (e.g., chitosan, silver ions, PEG, cationic peptides), anti-proliferative agents (e.g., sirolimus, methotrexate), antiapoptotic agents, antioxidants (e.g., ascorbic acid (vitamin C), glutathione (including N-acetyl-L cysteine), lipoic acid, alpha-tocopherol (vitamin E), ubiquinol as well as any synthetic analogues), immunosuppressive agents (e.g., cyclosporine, tacrolimus and etanercept), antibodies (polyclonal as well as monoclonal), antibiotics (e.g., amikacin, ciprofloxacin) and other drugs found to be beneficial for the health of the cochlea (e.g., salicylates, dizocilpine (MK801)).

Of the drugs listed above, the preferred antibiotic drug for application at a cochleostomy site is ciprofloxacin, and the preferred anti-inflammatory drug for application in the intra-cochlear region is dexamethasone. Additionally, neurotrophic factors, such as those listed above, may be beneficially applied to actively prevent the loss of spiral ganglion cells to potentially improve the performance of a cochlear implant.

It is to be understood that any suitable amount of a drug may be releasably carried in a drug-delivery element of the present invention. Additionally, drug-delivery elements in accordance with embodiments of the present invention may have various drug-delivery profiles.

In addition, embodiments of the present invention may be used for direct intra-cochlear drug delivery, which has significant potential advantages. For example, direct intra-cochlear drug delivery bypasses the blood-cochlea barrier allowing drugs to reach their intended targets more directly and utilizing lower doses of the drugs and less generalized application of drugs in the recipient. Additionally, drugs released into the perilymph compartment of the scala tympani may readily access the hair cells and the synaptic regions of the hair cells located in that area.

It is to be understood that one or more drugs may be disposed on or in a portion or substantially all of each drug-delivery element depending on the particular application. For example, it may be beneficial for a drug-delivery accessory to have a drug disposed in only a portion of the drug-delivery element, with the remaining portion of the drug-delivery element configured as a carrier or supporting member for delivery of the bioactive substance to the recipient.

As described above, in some embodiments, the drug-delivery spine may comprise silicone. Silicone in combination with one or more drugs has been known to swell, which may not be advantageous in some applications. However, in several embodiments of the present invention described above, the drug-delivery spine has an exposed outer surface, which allows the expansion of the spine to be accommodated.

Embodiments of the drug-delivery element of the present invention may be constructed as a woven mesh. In such embodiments, the threads of the woven mesh may be treated with one or more drugs during the fabrication of the mesh, or the mesh may be treated with one or more drugs subsequent to fabrication and prior to implantation with the implantable medical device.

According to a further embodiment of the present invention, the drug-delivery element may be constructed of a polymeric material, in which molecules or other components of a drug disposed are within the chemical structure of the drug-delivery accessory. One example of a polymeric material that may be used to construct an embodiment of a drug-delivery element of the present invention is silicone. One or more drugs may be disposed within the silicone drug-delivery element such that the drug(s) are released from the drug-delivery element.

In another embodiment, the drug-delivery element is configured to be bonded to the surface of the implantable medical device thereby eliminating the space or gap that may form between the drug-delivery element and the adjacent surface of the medical device component. The reduction and/or elimination of this gap reduces or eliminates the likelihood of bacterial growth between the two. In one embodiment, such bonding is performed in a substantially sterile field immediately prior to surgery. Alternatively, such bonding is performed after the medical device is implanted in the patient. In another embodiment, such bonding is performed during manufacturing, such as at one of the last few steps of manufacturing. In one embodiment, the bonding described above may be performed in a manner similar to any one of those described above in relation to the illustrative embodiment of FIGS. 2A and 2B.

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents. For example, in the description of the exemplary embodiments described above, the drug-delivery element is applied to a carrier member of a cochlear implant. It should be appreciated, however, that embodiments of the drug-delivery element of the present invention may be applied to other types of elongate components of implantable medical device. More broadly, aspects of the present invention may be implemented in implantable catheters. The present embodiments are, therefore, to be considered in all respects as illustrative and not restrictive.

What is claimed is:

1. An implantable medical device comprising:
an implantable elongate component; and
at least one elongate drug-delivery element releasably carrying at least one drug and configured to be mounted to the elongate component such that the longitudinal axis of the drug-delivery element is substantially parallel with a longitudinal axis of the elongate component.

2. The device of claim 1, wherein the drug-delivery element is formed of a solid material.

3. The device of claim 2, wherein the elongate component is configured to assume a fully-curved configuration substantially matching the shape of a modiolus of a cochlea, and wherein the drug-delivery element is resorbable and is configured to bias the elongate component into a less-curved configuration when mounted to the elongate component.

4. The device of claim 1, wherein the drug-delivery element is formed of a semisoft material.

5. The device of claim 1, wherein the drug-delivery element is resorbable.

6. The device of claim 5, wherein the elongate component is configured to assume a pre-curved configuration substantially matching the shape of a modiolus of a cochlea, and wherein the drug-delivery element is pre-curved and is configured to bias the elongate component into a radius of curvature greater than that of the pre-curved configuration when mounted to the elongate component.

7. The device of claim 1, wherein the elongate component comprises at least one recess and the drug-delivery element is configured to be mounted in the recess.

8. The device of claim 7, wherein the drug-delivery element is configured to be mechanically locked in the recess when the drug-delivery element is mounted in the recess.

9. The device of claim 8, wherein the drug-delivery element is dimensioned such that the drug-delivery element forms an interference fit within the recess when the drug-delivery element is mounted in the recess.

10. The device of claim 7, wherein the drug-delivery element is dimensioned such that the drug-delivery element partially fills the recess when mounted in the recess.

11. The device of claim 7, wherein the at least one recess comprises a plurality of recesses, the at least one drug-delivery element comprises a plurality of drug-delivery elements, and the plurality of drug-delivery elements are configured to be mounted in the plurality of recesses, respectively.

12. The device of claim 11, wherein two of the plurality of drug-delivery elements releasably carry different drugs.

13. The device of claim 1, wherein the elongate component comprises a dorsal side, a ventral side and a transition region, wherein the elongate component has a first thickness between the dorsal and ventral sides of the elongate component on a first side of the transition region and a second thickness substantially smaller that the first thickness between the dorsal and ventral sides of the elongate component on a second side of the transition region.

14. The device of claim 13, wherein the elongate component comprises a lowered surface having a substantially flat cross section extending away from the transition region on the second side of the transition region.

15. A kit for an implantable drug-delivery system, the kit comprising:
an implantable elongate component;
at least one first drug-delivery element; and
at least one second drug-delivery element, wherein each of the first and second drug-delivery members releasably carries at least one drug and is configured to be mounted along a length of the elongate component such that the longitudinal axis of the drug-delivery element is substantially parallel with a longitudinal axis of the elongate component.

16. The kit of claim 15, wherein the first drug-delivery element releasably carries a first drug and the second drug-delivery element releasably carries a second drug different than the first drug.

17. The kit of claim 15, wherein the first drug-delivery element is formed of a solid material and the second drug-delivery element is formed of a semisoft material.

18. The kit of claim 15, wherein the first and second drug-delivery elements releasably carry different dosages of the same drug.

19. The kit of claim 15, wherein the elongate component comprises an elongate recess substantially parallel with the longitudinal axis of the elongate component, the first drug-delivery element is configured to be mounted in the recess so as to substantially fill the recess, and the second drug-delivery element is configured to be mounted in the recess so as to partially fill the recess.

20. The kit of claim 15, wherein the first drug-delivery element is resorbable and the second drug-delivery element is non-resorbable.

21. The kit of claim 15, wherein the first and second drug-delivery elements have different cross-sectional shapes.

22. An implantable elongate drug-delivery element for an implantable elongate component of an implantable medical device, the elongate component comprising an elongate recess, the drug-delivery element comprising:
an elongate substrate configured to be releasably mounted in the elongate recess such that the longitudinal axis of the drug-delivery element is substantially parallel with the longitudinal axis of the elongate component; and
a drug releasably carried by the substrate.

23. The drug-delivery element of claim 22, wherein the substrate is formed of a solid material.

24. The drug-delivery element of claim 22, wherein the substrate is resorbable.

25. The drug-delivery element of claim 22, wherein the substrate is configured to form an interference fit within the recess.

26. The drug-delivery element of claim 22, wherein the substrate has a substantially circular cross-section.

27. The drug-delivery element of claim 22, wherein the substrate has a trapezoidal cross-section.

28. The drug-delivery element of claim 22, wherein the substrate has a rectangular cross-section.

29. A method of using an implantable medical device comprising an implantable elongate component and at least one drug-delivery element releasably carrying at least one drug, the method comprising:
mounting the drug-delivery element along a length of the elongate component such that the longitudinal axis of the drug-delivery element is substantially parallel with a longitudinal axis of the elongate component; and
implanting the elongate component in a recipient subsequent to mounting the drug-delivery member.

30. The method of claim 29, wherein the elongate component comprises an elongate recess substantially parallel with a longitudinal axis of the elongate component, and wherein mounting the drug-delivery element comprises:
at least partially filling the recess with a paste releasably carrying the at least one drug.

31. The method of claim 30, wherein at least partially filling the recess with the paste comprises:
dispensing the paste into the recess via a syringe.

32. The method of claim 29, wherein the elongate component comprises an elongate recess substantially parallel with a longitudinal axis of the elongate component, and wherein mounting the drug-delivery element comprises:
at least partially filling the recess with ultraviolet (UV) curable silicone mixed with the at least one drug; and
exposing the UV curable silicone to UV light.

33. The method of claim 29, wherein mounting the drug-delivery element comprises:
bonding the drug-delivery element to the elongate component.

34. The method of claim 29, wherein the elongate component comprises an elongate recess substantially parallel with a longitudinal axis of the elongate component, and wherein mounting the drug-delivery element comprises:
pressing the drug-delivery element into the recess so as to mechanically lock the drug-delivery element in the recess.
